(19) United States
(12) Patent Application Publication
(10) Pub. No.: US 2016/0002278 A1
(43) Pub. Date: Jan. 7, 2016

(54) Pharmaceutical Compositions for Rectal Administration

(71) Applicant: CIPLA LIMITED, Mumbai (IN)

(72) Inventors: Shrinivas Madhukar PURANDARE, Mumbai (IN); Geena MALHOTRA, Mumbai (IN)

(73) Assignee: Cipla Limited, Mumbai (MH)

(21) Appl. No.: 14/770,315

(22) PCT Filed: Mar. 7, 2014
(86) PCT No.: PCT/GB2014/050678

§ 371 Date: Aug. 25, 2015

(30) Foreign Application Priority Data
Mar. 8, 2013 (IN) 711/MUM/2013

(51) Int. Cl. C07H 17/08; A61M 31/00; A61K 9/00; A61K 45/06; A61K 31/7048; A61K 9/12

(52) U.S. Cl. CPC C07H 17/08; A61K 31/7048; A61K 9/124; A61K 9/0031; A61K 45/06; A61M 31/00

(57) ABSTRACT

The present invention relates to pharmaceutical compositions for rectal administration comprising fidaxomicin and to a process for preparing the pharmaceutical compositions for rectal administration. The invention also relates to an aerosol canister comprising a foamable pharmaceutical composition comprising fidaxomicin for rectal administration and to the treatment or maintenance of remission of infections such as diarrhea caused by Clostridium difficile.

### CROSS-REFERENCE TO RELATED APPLICATIONS

This application is a filing under 35 U.S.C. 371 of International Application No. PCT/GB2014/050678 filed Mar. 7, 2014, entitled “Pharmaceutical Compositions for Rectal Administration” which claims priority to Indian Patent Application No. 71 l/MUM/2013 filed Mar. 8, 2013, which applications are incorporated by reference herein in their entirety.

### FIELD OF INVENTION

The present invention relates to pharmaceutical compositions for rectal administration comprising fidaxomicin, a process for preparing the pharmaceutical compositions for rectal administration and their use in the treatment of infections caused by Clostridium difficile.

### BACKGROUND AND PRIOR ART

Clostridium difficile is gram-positive toxin producing bacteria. It invades the intestinal tracts of patients whose normal intestinal flora is suppressed due to treatment with broad-spectrum antibiotics. The bacterial toxins cause varying degrees of damage to the large intestinal (i.e., colonic) epithelium and cause a spectrum of illnesses, ranging from mild diarrhea to severe colitis. Because antibiotic treatment induces the onset of Clostridium difficile disease, the associated syndromes are referred to as antibiotic-associated diarrhea and colitis. It is becoming an increasing problem, with the emergence of hyper-virulent bacterial strains and with an geriatric population who are most affected by it. It causes around 15,000 deaths in the US each year according to the Centers for Disease Control. Further, Clostridium difficile is a spore forming bacteria that, when under attack, goes into a vegetative state if it is not killed and will keep producing spores. In the absence of gut flora, the spore production is accelerated and the chances for re-occurrence increases.

Various therapies have been developed to treat or specifically attack Clostridium difficile like for example U.S. Pat. No. 6,969,520 provides active and passive immunization methods for preventing and treating Clostridium difficile infections, which involve percutaneous administration of Clostridium difficile toxin-neutralizing polyclonal immune globulin, Clostridium difficile toxoids or combinations thereof.

US20130004561 provides an antibody composition comprising ovine antibodies, for use in the prevention or treatment of Clostridium difficile infection, wherein the antibodies bind to a Clostridium difficile toxin and wherein said prevention or treatment is by oral delivery of the antibody composition.

US20130022575 provides one or more systems and methods for treating diseases including Clostridium difficile and Crohn's disease by introducing a mixture of pure cultures of viable bacteria into the gastrointestinal tract.

Although administration via the peroral route is the most commonly targeted goal of new drug and dosage form research and development, oral administration is not always feasible or desirable. Further certain patient populations notably pediatric patients and geriatric patients, and those with swallowing problems, are often difficult to treat with oral tablets and capsules. Additionally, treatment of some diseases is best achieved by direct administration near the affected area, particularly with diseases involving colon or anorectal tissues. Although oral administration may be used for drugs targeted for some of these diseased tissues, exposure of the entire body compartment to the administered drug may lead to adverse effects. Some animal study data of fidaxomicin showed that oral administration of fidaxomicin in addition to reaching the site of action and exhibiting the desired effects was also distributed to other vital organs such as lungs producing dark red discoloration of the lungs and lymph nodes.

Rectal drug administration is amenable, however, to both local and systemic drug delivery. It has been effectively utilized to treat local diseases of the anorectal area as well as to deliver drugs systemically as an alternative to oral administration. Some advantages of this targeted delivery which includes, but not limited to, treatment of large surface area, ability to bypass first-pass metabolism as well as prolonged residence time makes this route more promising for delivery of locally acting drugs.

Fidaxomicin, formerly called OPT-80, is the first experimental drug in a new class of narrow spectrum macrocyclic antibiotic drugs. While many antibiotics aim to stop the growth of infectious bacteria, fidaxomicin induces the death of Clostridium difficile by inhibiting a bacterial enzyme called RNA polymerase. Fidaxomicin has a narrow-spectrum of activity believed to selectively eradicate Clostridium difficile with minimal disruption to the normal intestinal flora, leaving healthy flora unharmed.

Fidaxomicin is chemically known as Oxacyclooctadeca-3,5,9,13,15-pentaen-2-one, 3-[[[6-deoxy-4-O-(3,5-dichloro-2-ethyl-4,6-dihydroxybenzoyl)-2-O-methyl-b-D-mannopyranosyl]oxy]methyl]-12-[[6-deoxy-5-C-methyl-4-O-(2-methyl-1-oxopropyl)-b-D-lyxo-hexopyranosyl]oxy]-1-ethyl-8-hydroxy-18-[(1R)-1-hydroxyethyl]-9,13,15-trimethyl(3E,5E,8S,9E,1S,12R,13E,15E,18S marketed as Dificid® and has following structure

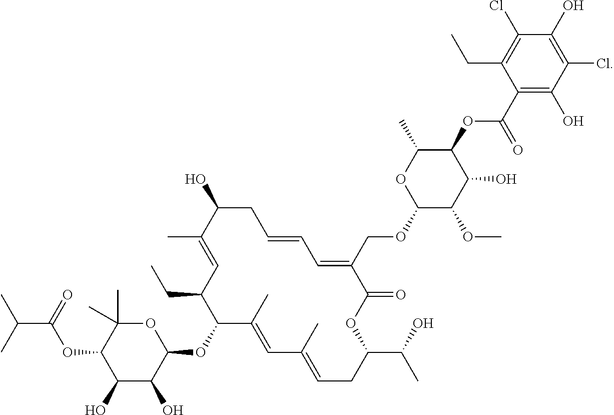 [SMILES: CCc1c(Cl)c(O)c(Cl)c(O)c1C(=O)O[C@H]1[C@H](O)[C@H](OC)[C@H](OC/C2=C\C=C\C[C@H](O)/C(C)=C/[C@H](CC)[C@@H](O[C@@H]3OC(C)(C)[C@@H](OC(=O)C(C)C)[C@H](O)[C@@H]3O)/C(C)=C/C(C)=C/C[C@@H]([C@@H](C)O)OC2=O)O[C@@H]1C]

U.S. Pat. No. 3,978,211 discloses fidaxomicin specifically characterized by the physiochemical properties along with the process for manufacturing the same.

U.S. Pat. No. 7,378,508 discloses polymorphic forms and the compositions of fidaxomicin for use in the treatment of diarrhea caused by Clostridium difficile.

U.S. Pat. No. 7,863,249 discloses a pharmaceutical composition comprising a therapeutically effective amount of a polymorphic form of fidaxomicin in the form of film coated tablets.

U.S. Pat. No. 7,906,489 discloses a method of treating diarrhea caused by Clostridium difficile gastrointestinal infection comprising orally administering a therapeutically effective amount of fidaxomicin to a human being in need thereof.

Fidaxomicin has a high molecular weight (1,058 g/mol) and exhibits low aqueous solubility (10-20 g/mL at neutral pH) that contributes to the overall poor bioavailability (1%) after oral administration.

Generally, the topical delivery of active agents via the rectal route can be achieved by using suppositories, enemas, ointments, creams or foams. Suppositories are the most common rectal dosage forms. The suppository bases are generally fatty in nature, but water-soluble or water-miscible bases can also be utilized. In order to achieve a desirable bioavailability the active ingredient should come in contact with the rectal or colonic mucosa.

Rectal dosage forms such as ointments and creams create an environment which is not favorable to the respiration of the wound. Moreover, there may be likelihood of experiencing pain and irritation during the application of rectal ointments and creams, particularly to the abraded, wounded or inflamed mucosa of the rectum or colon. Rectal foams are less commonly preferred as compared to any other rectal dosage forms. However, rectal enema and foams exhibit better spreadability since they enable the drug to reach the distal part of the intestinal regions.

Amongst the known rectal dosage forms rectal enemas and foams exhibit better spreadability effect, thus enabling them to reach the distal intestinal regions. However, rectal enemas can be uncomfortable for the patient, because its administration can stimulate the urge to defecate, and this, combined with the need for enema retention, can cause patient embarrassment and discomfort. Complications may include leakage, bloating, irritation, bleeding, swelling, or prolapse of the rectal tissue. Further rectal enemas are difficult to be self administered by a patient.

Although rectal foams provide various advantages such as better spreadability in the surrounding tissues as compared to the other rectal dosage forms, their formulation requires a specific balance between the foam-forming excipients. It may be possible that anyslight deviation of such foam-forming excipients may result in a foam which is unstable or not formed at all, especially when the administration is to occur via a small diameter applicator nozzle.

Most rectal foams are known to contain corticosteroids, although rectal foams have been designed to deliver antiseptics, antifungal agents, anti-inflammatory agents, local anesthetic agents, skin emollients, and protectants. However, the prior art does not disclose rectal foam formulations or compositions specifically for treatment of infections caused by Clostridium difficile.

Thus, there exists a need to develop a topical pharmaceutical composition of fidaxomicin which is suitable for rectal administration.

### OBJECT OF THE INVENTION

An object of the present invention is to provide a pharmaceutical composition suitable for rectal administration in the form of a foam comprising fidaxomicin.

Another object of the present invention is to provide a foamable pharmaceutical composition for rectal administration comprising fidaxomicin.

Another object of the present invention is to provide a pharmaceutical composition for rectal administration in the form of a foam comprising fidaxomicin which exhibits better spreadability.

Yet another object of the present invention is to provide a stable pharmaceutical composition for rectal administration in the form of a foam comprising fidaxomicin.

Yet another object of the present invention is to provide a pharmaceutical composition for rectal administration in the form of a foam comprising fidaxomicin which remains effective even after intestinal evacuation by the patient.

Yet another object of the present invention is to provide a process of manufacturing a pharmaceutical composition comprising fidaxomicin, which is suitable for rectal administration in the form of a foam.

Yet another object of the present invention is to provide a device comprising a pharmaceutical composition comprising fidaxomicin, which is capable of forming a foam.

Yet another object of the present invention is to provide a method of treating or maintenance of remission of Clostridium difficile infection by administering a pharmaceutical composition for rectal administration in the form of a foam comprising fidaxomicin to patients in need thereof.

Yet another object of the present invention is to provide a pharmaceutical composition for rectal administration in the form of a foam comprising fidaxomicin for the use in the treatment of Clostridium difficile associated diarrhea.

### SUMMARY OF THE INVENTION

According to one aspect of the present invention there is provided a pharmaceutical composition in the form of a foam for rectal administration comprising fidaxomicin and optionally one or more pharmaceutically acceptable excipients.

According to another aspect of the present invention there is provided a foamable pharmaceutical composition for rectal administration comprising fidaxomicin and optionally one or more pharmaceutically acceptable excipients.

According to another aspect of the present invention there is provided a stable foamable pharmaceutical composition for rectal administration comprising fidaxomicin.

According to another aspect of the present invention, there is provided a pharmaceutical composition for rectal administration in the form of a foam comprising fidaxomicin wherein the total daily dose of the fidaxomicin is less than 400 mg.

According to another aspect of the present invention, there is provided a pharmaceutical composition for rectal administration in the form of a foam comprising fidaxomicin for once or twice a day administration.

According to another aspect of the present invention there is provided a process of manufacturing a pharmaceutical composition for rectal administration in the form of a foam comprising fidaxomicin, the process comprises: heating a vehicle and adding an emulsifier to the vehicle, optionally along with other pharmaceutically acceptable excipients followed by adding fidaxomicin to obtain a blend; optionally, filling the blend into a container and charging it with a propellant.

According to another aspect of the present invention there is provided an aerosol canister for a pharmaceutical composition for rectal administration in the form of a foam comprising fidaxomicin, the canister comprising a housing containing under pressure the pharmaceutical composition; means for measuring a metered dose of the composition from the canister for administration to a patient in need thereof; and optionally comprising an applicator device for rectal administration.

According to another aspect of the present invention there is provided a method of treating or maintenance of remission of Clostridium difficile infection by administering a pharmaceutical composition for rectal administration in the form of a foam comprising fidaxomicin to patients in need thereof.

According to a further aspect of the present invention there is provided a pharmaceutical composition for rectal administration in the form of a foam comprising fidaxomicin for use in the treatment of infection caused by Clostridium difficile.

According to yet another aspect of the present invention there is provided a method of treating, or alleviating an anal disorder comprising administering a pharmaceutical composition for rectal administration in the form of a foam fidaxomicin to a subject in need thereof.

### DETAILED DESCRIPTION OF THE INVENTION

Certain medicaments require local or topical administration. Thus, some medicaments require administration by oral route or rectal route, the latter applying when the aim is to treat pathological states of the rectum or the like.

Fidaxomicin is a 18-membered macrocyclic antimicrobial agent also know as Tiacumicins or OPT-80 (which is composed almost entirely of the R-Tiacumicin B). Currently, fidaxomicin is commercially available as oral tablets (Dificid® tablets, 200 mg/twice a day) to be administered for 10 days. Fidaxomicin is minimally absorbed from the gastrointestinal tract following oral (PO) administration due to its poor permeability and solubility properties. Further, oral administration of fidaxomicin causes adverse effects such as hives, difficulty in breathing, swelling of face, lips, tongue or throat.

Therefore, there is a need to seek release of fidaxomicin at the actual site of action via rectal route so as to address the permeability and solubility issues.

Further, during formulation of such rectal foams it is desirable to maintain a balance between the foam forming excipients and it is believed that any slight deviation of such foam-forming excipients may result in a foam which is unstable or not formed at all.

The present invention thus provides a pharmaceutical composition suitable for rectal administration comprising fidaxomicin and optionally one or more pharmaceutically acceptable excipients and which exhibits optimum stability.

The present invention also provides a pharmaceutical composition suitable for rectal administration in the form of a foam comprising fidaxomicin.

The present invention also provides a foamable pharmaceutical composition for rectal administration comprising fidaxomicin.

As used herein, the term “fidaxomicin” or “active agent” is used in a broad sense to include not only “fidaxomicin” per se but also its pharmaceutically acceptable derivatives thereof. Suitable derivatives include pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

The amount of fidaxomicin in the pharmaceutical composition for rectal administration according to the present invention is preferably in the range from about 0.01% w/w to about 30% w/w of the total weight of the pharmaceutical composition, more preferably from about 0.5% w/w to about 25% w/w of the total weight of the pharmaceutical composition.

The pharmaceutical composition according to the present invention refers to a foamable pharmaceutical composition for rectal administration comprising fidaxomicin and also refers to a pharmaceutical composition for rectal administration in the form of a foam comprising fidaxomicin.

The present invention provides a pharmaceutical composition for rectal administration comprising fidaxomicin with one or more pharmaceutically acceptable excipients and filled in a compressed gas container, that upon valve actuation, emits a fine dispersion of liquid and/or solid materials in a gaseous medium.

The pharmaceutical compositions for rectal administration of the present invention have the advantages of being easy to apply, less dense, come in contact with the mucous without any latency time, more retention time at site of action, less rigid and adapt to contours, less irritability and exhibit more spreadability as compared to other rectal dosage forms. Another advantage is ease of use and patient compliance.

The pharmaceutical compositions for rectal administration of the present invention may be formulated with appropriate excipients so as to provide emollient or drying effect to the rectal mucosa.

The pharmaceutical compositions are suitable for rectal and/or colonic administration and/or administration to terminal ileum of a patient for the treatment, or maintenance of remission of infection caused by Clostridium difficile.

Suitable excipients such as, but not limited to, vehicle, preservatives, surfactants, emulsifiers, mineral oils, propellants, thickening agents, lubricants, preservatives, pH adjusting agents, chelating agents, emollients and/or humectants, permeation enhancers, suspension-forming agents or mucoadhesive agents or combinations thereof, may be used for formulating the pharmaceutical compositions for rectal administration according to the present invention.

The vehicle may include an aqueous, non-aqueous or a hydro-alcoholic vehicle. Suitable aqueous vehicles which are compatible with the rectal and colonic mucosa, may comprise water soluble alkanols selected from, but not limited to, ethanol, polyalcohols such as a propylene glycol, glycerol, polyethyleneglycol, polypropylene glycol, propylene glycol glyceryl esters and combinations thereof. Non-aqueous vehicles which may be employed in the pharmaceutical rectal foam compositions of the invention, include but not limited to vegetable oils, such as olive oil; injectable organic esters, such as ethyl oleate and combinations thereof.

The vehicle may also be selected from highly hydrophilic organic substances to allow the surfactant to perform its foaming action, which however preferably should not be inhibited by the other substances present in the compositions, such as the active principles and their stabilizers, whereas the specific adjuvants (such as foam consistency correctors) are preferably chosen from those with strong hydrophilic and lipophilic characteristics.

The vehicle may be present in an amount in the range from about 10% w/w to about 95% w/w of the total weight of the pharmaceutical composition, preferably from about 10% w/w to about 90% w/w of the total weight of the pharmaceutical composition, more preferably from about 20% to about 85% w/w of the total weight of the pharmaceutical composition.

Suitable surfactants which may be employed in the pharmaceutical composition for rectal administration of the present invention includes, but not limited to anionic surfactants, non-ionic surfactants, cationic surfactants, and amphoteric surfactants.

Anionic surfactants may include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinate, disodium lauryl sulfosuccinate, diammonium lauryl sulfosuccinate, tetrasodium N-(1,2-dicarboxyethyl)-N-octadecylsulfosuccinate, diamyl ester of sodium sulfosuccinic acid, dihexyl ester of sodium sulfosuccinic acid, dioctyl esters of sodium sulfosuccinic acid, docusate sodium, and combinations thereof.

Nonionic surfactants may include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethylene oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and combinations thereof.

Amphoteric surfactants may include, but are not limited to, sodium N-dodecyl--alanine, sodium N-lauryl- -iminodipropionate, myristoamphoacetate, lauryl betaine, lauryl sulfobetaine, sodium 3-dodecyl-aminopropionate, sodium 3-dodecylaminopropane sulfonate, sodium lauroamphoacetate, cocodimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and combinations thereof.

Cationic surfactants may include, but are not limited to, behenyl trimethyl ammonium chloride, bis(acyloxyethyl)hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowedimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquatemium, stearalkonium chloride, sterayl dimethylbenzyl ammonium chloride, stearyl trimethyl ammonium chloride, trimethylglycine, and combinations thereof.

The surfactant may be present in an amount of from about 0.1% to about 15% w/w of the total weight of the pharmaceutical composition; more preferably, in an amount from about 0.1% to about 12% w/w of the total weight of the pharmaceutical composition.

Suitable thickening agents or viscosity modifying agents which may be employed in the pharmaceutical composition for rectal administration include, but are not limited to, carboxymethyl cellulose, polyoxyethylene-polyoxypropylene copolymers, xanthan gum, agar, guar gum, hydroxyethyl cellulose, hydroxypropyl cellulose, methyl cellulose and combinations thereof.

The thickening agentmay be present in an amount from about 0.01 to about 3% (w/w) of the total weight of the pharmaceutical composition for rectal administration.

It will be appreciated by the person skilled in the art that surfactants selected may either provide an emulsifying action or provide a foam-stabilizing action. The surfactant(s) is desirably selected such that it remains compatible with the rectal and colonic mucosa and preferably present in an amount which achieves the desired pharmaceutical effect but which does not give rise to problems of irritation.

Propellants may be used in the pharmaceutical composition for rectal administration to accomplish a foaming effect. The propellant may be selected according to known principles for preparing a foamable composition of the aerosol type packed in a pressurized container and suitable for a rectal application. The propellant may be any suitable, pharmaceutically acceptable gas such as a low molecular weight hydrocarbon e.g. isobutane, n-butane, propane, CFC, hydrocarbons; chlorofluorocarbons (CFCs); hydrochlorofluorocarbons (HCFCs); hydrofluoroalkanes (HFAs) such as HFA 134a and HFA 227; and combinations thereof. Preferably, the propellant comprises a mixture of n-butane, isobutane, propane.

The propelling properties can vary depending on the type and quantity of propellant used and, consequently, the foam can reach more or less distant regions of the large intestine.

The propellant may be present in an amount from about 0.05 to about 20% w/w, preferably from about 0.5 to about 20% w/w, of the composition, most preferablyfrom about 1% to about 10% of the composition.

Additionally, liquefied nitrogen may be present as pressurizing agent to obtain the required number of doses.

In a preferred embodiment, the pharmaceutical compositions for rectal administration comprises fidaxomicin, at least one propellant, at least one vehicle, at least one emulsifier and/or surfactant and optionally other pharmaceutically acceptable excipients.

Further, the pharmaceutical compositions according to the present invention for rectal administration may be stable non-aqueous (anhydrous) foams, stable aqueous foams, evanescent or quick breaking non-aqueous foams or evanescent or quick breaking aqueous foams.

Further, the pharmaceutical compositions according to the present invention for rectal administration may comprise at least one additional active ingredient suitable for rectal administration.

Additional active ingredients may be selected from, but not limited to one or more of anti-inflammatory agents, steroids (e.g. corticosteroids), additional antibiotics, anti-fungal agents, analgesics, or anti-neoplastic agents, antivirals, anaesthetics and combinations thereof.

Examples of suitable antibiotics includes, but not limited to: dapsone, chloramphenicol, neomycin, cefaclor, cefadroxil, cephalexin, cephradine, erythromycin, clindamycin, lincomycin, amoxicillin, ampicillin, bacampicillin, carbenicillin, dicl oxacillin, cyclacillin, picloxacillin, hetacillin, methicillin, nafcillin, penicillin, polymyxin, tetracycline, amphotericin-b, candicidin, dermostatin, filipin, fungichromin, hachimycin, hamycin, lucensomycin, mepartricin, natamycin, nystatin, pecilocin, perimycin, azaserine, griseofulvin, oligomycins, neomycin undecylenate, pyrroinitrin, siccanin, tubercidin, viridin, picloxacillin, hetacillin, methicillin, nafcillin, penicillin, polymyxin, or tetracycline and combinations thereof.

Examples of suitable anitfungal agents includes but not limited to: allylamines such as butenafine, naftifine, imidazoles such as bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole, triazoles such as fluconazole, itraconazole, saperconazole, terconazole, and others such as acrisorcin, amorolf[iota]ne, biphenamine, bromosalicylchloranilide, buclosamide, calcium propionate, chlophenesin, ciclopirox, cloxyquin, coparaff[iota]nate, diamthazole, dihydrochloride, exalamide, flucytosine, halethazole, hexetidine, Ioflucarban, nifuratel, potassium iodide, propionates, propionic acid, pyrithione, salicylanilide, sulbentine, tenonitrozole, triacetin, ujothion, undecylenic acid and combinations thereof.

Antifungal agents may also include, for example, polyenes such as amphotericin-b, candicidin, dermostatin, filipin, fungichromin, hachimycin, hamycin, lucensomycin, mepartricin, natamycin, nystatin, pecilocin, perimycin, azaserine, griseofulvin, oligomycins, neomycin undecylenate, pyrroinitrin, siccanin, tubercidin, viridin, allylamines such as butenafine, naftifine, imidazoles such as bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole, triazoles such as fluconazole, itraconazole, saperconazole, terconazole, acrisorcin, amorolf[iota]ne, biphenamine, bromosalicylchloranilide, buclosamide, calcium propionate, chlophenesin, ciclopirox, cloxyquin, coparaff[iota]nate, diamthazole, dihydrochloride, exalamide, flucytosine, halethazole, hexetidine, Ioflucarban, nifuratel, potassium iodide, propionates, propionic acid, pyrithione, salicylanilide, sulbentine, tenonitrozole, triacetin, ujothion, or undecylenic acid and combinations thereof.

The other therapeutic agent can include steroid or a non-steroidal antiinflammatory agent. Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflum[iota]c acid, tolfenamic acid, diflurisal, flufenisal, piroxicam, sudoxicarn, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and combinations thereof.

Examples of suitable corticosteroid includes but not limited to: hydrocortisone, i.e., 11-17-21-trihydroxypregn-4-ene-3,20-dione or Cortisol, Cortisol acetate, hydrocortisone phosphate, hydrocortisone 21-sodium succinate, hydrocortisone tebutate, corticosterone, corticosterone acetate, cortisone, cortisone acetate, cortisone 21B-cyclopentanepropionate, cortisone phosphate, triamcinolone hexacetonide, dexamethasone phosphate, desonide, betamethasone dipropionate, mometasone furate and combinations thereof.

The corticosteroid and topical anesthetic may be employed together in the pharmaceutical rectal foam composition along with fidaxomicin.

For inflammation, preferred treatments for use in combination therapy with the compositions of the present invention include, but not limited to naproxen sodium, flurbiprofen, diclofenac sodium, misoprostil, valdecoxib, diclofenac potassium, celecoxib, sulindac, oxaprozin, salsalate, difhmisal, naproxen sodium, piroxicam, indomethacin and Indocin SR, etodolac, meloxicam, ibuprofen, naproxen, ketoprofen, nabumetone, tolmetin sodium, choline magnesium trisalicylate, and rofecoxib.

Antineoplastic agents may also be included in the pharmaceutical rectal foam composition of the present invention along with the Fidaxomicin. Suitable antineoplastic agents include, but not limited to: vincristine, vinblastine, vindesine, busulfan, chlorambucil, spiroplatin, cisplatin, carboplatin, methotrexate, adriamycin, mitomycin, bleomycin, arabinosyl adenine, mercaptopurine, mitotane, procarbazine, dactinomycin (antinomycin D), daunorubicin, doxorubicin hydrochloride, taxol, plicamycin, aminoglutethimide, estramustine, flutamide, leuprolide, megestrol acetate, tamoxifen, testolactone, trilostane, amsacrine (m-AMSA), asparaginase (L-asparaginase), etoposide, and interferon a-2a and 2b and combinations thereof.

Antiviral agents may also be included in the topical foam composition of the present invention along with the Fidaxomicin. Suitable antiviral agents include, but not limited to: acyclovir, amantadine, azidothymidine, ribavirin or vidarabine. In any case where pain in a component of the target disorder, the other therapeutic agent can be an analgesic. Useful analgesics include, but are not limited to, phenacetin, butacetin, acetaminophen, nefopam, acetoamidoquinone, and combinations thereof.

A topical anesthetic may be present in the pharmaceutical rectal foam composition of the invention. For instance, the topical anesthetic may include, but not limited to dibucaine, lidocaine, pramoxine, benzocaine, tetracaine and combinations thereof. In general, the topical anesthetic may be present in any amount which is effective in the practice of the treatment of infection casued by Clostridium difficile, namely diarrhea.

The pharmaceutical composition for rectal administration of the present invention may also comprise the actives in nanosize form.

The term “nanosize” as used herein refers to particles of actives having an average particle size of less than or equal to about 2000 nm, preferably less than or equal to about 1000 nm.

The pharmaceutical composition for rectal administration according to the present invention is preferably packed in a suitable pressurized dispensing canister of the aerosol type well known in the art such as an aluminium canister. Each canister is sealed with a suitable foam dispensing valve. Any valve or nozzle/valve assembly which provides a means for releasing the foam from the canister and provides foam which is suitable for use in the present invention may be used. The pharmaceutical rectal foam exhibits superior properties. The advantages associated with the pharmaceutical rectal foam composition according to the present invention is that better results may be obtained in combating the disease and either a lower dosage of the active ingredient or less dosages per day may be necessary to obtain similar results when compared with prior art compositions. For instance, the increased spreadability of the foam together with the longer exposure time to the drug will result in optimal local effect at the target site. Also, the rectal foam of the present invention is expected not to cause extra irritation of the inflamed target mucosa. Due to these superior properties of the foam, the present invention may represent a valuable alternative to previously known dosage forms used in the treatment of infections caused by Clostridium difficile.

The pharmaceutical composition of the present invention for rectal administration is presented in a suitable dispensingcanister, for example an aluminium aerosolcanister, fitted with a suitable metered or un-metered valve. Such canisters are well known in the art. Where desired, the canister can be fitted or supplied together with an applicator device for insertion into the rectum to ensure more efficient administration of the rectal foam.

The dispensing canister may be in the form of coated aluminium cans to prevent corrosion, such as epoxy-coated cans. At the time of application, the mixing of the ingredients with propellant may be ensured by shaking, or with the aid of a mixing bead. The can may be arranged for either “upside down” spraying with the valve at the bottom, or the can have a dip tube so that the foam can be sprayed while the can is upright with the valve at the top.

Additionally the dispensing canister, may comprise a metered pump dome which can be fixed onto the canister, preferably on the top position, which ensures an optimum amount of the dose being dispensed.

During the use, the dispensing valve of the can allows rapid expansion of the propellant, which triggers and enhances the foaming action of the surfactant, which thus emerges to entrain the medicated liquid in the form of a rectal foam.

The propellant expansion energy is absorbed mainly in forming the foam, thus allowing rectal application without risk.

According to the present invention, the rectal foam may be generated at the moment of therapeutic application. Hence the known formulation and dispensing technology used in the state of the art applicable to foam cans, for example in cosmetics, may be therefore suitable to obtain a rectal foam.

Further, the main disadvantage of rectal foams is their low density, which is typically of the order of 0.1 g/l which does not allow higher amounts of an active principle to be administered. This low density makes it necessary to administer large amounts of foam which is problematical in view of the limited volume of the rectum (between about 50 ml and 400 ml).

The inventors of the present invention have optimized the ability to achieve the desired efficacy with the administration of a minimal volume of 0.5 g to 10 g of the rectal foam according to the present invention.

The present invention further provides a process of manufacturing the pharmaceutical composition for rectal administration comprising fidaxomicin, the process comprising: 1) heating a vehicle and adding an emulsifier to the heated vehicle; 2) adding a preservative and a chelating agent 3) adding the active agent fidaxomicin to it under stirring to obtain a suspension; and optionally 4) filling the solution in a canister and charging it with a propellant.

The vehicle, emulsifier, preservative, chelating agent and the propellant as defined herein above maybe used in the process according to the present invention.

The active agent is solubilized or suspended in a suitable liquid vehicle containing a emulsifier. The liquid comprising the active agent and emulsifier is filled in an atomizer can which is then sealed by a dispensing valve and further pressurized by feeding a suitable quantity of propellant through the valve.

It will be appreciated by the person skilled in the art that the pharmaceutical rectal foam composition comprising fidaxomicin may further comprise one or more pharmaceutical excipients, selected from, but are not limited to: emollient or humectants, pH adjusting agent, emulsifiers, foaming agents, fatty alcohol, preservative, chelating agents, antioxidants, suspending agents, thickening agents, permeation enhancers, occlusive agents, colorants and fragrances or combinations thereof.

Examples of suitable pH adjusting agents may be selected from, but not limited to, sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof, preferably triethanolamine is used.

The pharmaceutical composition for rectal administration according to the present invention may comprise a suitable pH adjusting agent to adjust the pH in the range from approximately 4 to 8.

Examples of the emulsifiers that can be used in the pharmaceutical composition of the present invention for rectal administration include polysorbates (Tween® 20, Tween® 40, Tween® 60, Tween® 80, Nonylphenol Polyethylene Glycol Ethers, (alkylphenol-hydroxypolyoxyethylene), Poly(oxy-1,2-ethanediyl), alpha-(4-nonylphenol)-omega-hydroxy-, Nonylphenol Polyethylene Glycol Ether mixtures, phenoxypolyethoxyethanols and polymers thereof such as Triton®, Poloxamer®, Spans®, Tyloxapol®, different grades of Brij, sodium dodecyl sulfate and the like or combinations thereof. Preferably, the emulsifiers that are used in the pharmaceutical composition of the present invention for rectal administration include emulsifying waxes such as those described in the U.S. National Formulary (USNF) and ‘Martindale’ such as cetyl alcohol, steryl alcohol, cetosteryl alcohol, cetomacrogols and the like or combinations thereof. An emulsifying wax may be incorporated in the pharmaceutical rectal composition of the present invention in order to stiffen the foam.

The amount of emulsifier in the composition is preferably from 0.5% to 10% w/w based on the total weight of the pharmaceutical composition.

Examples of suitable emollients and/or humectants which may be employed in the pharmaceutical composition of the present invention for rectal administration include, but not limited to, polyhydric alcohols such as glycols, and polysaccharides, such as ethylene glycol, propylene glycol, butylene glycol, diethylene glycol, dipropylene glycol, glycerin, diglycerin, sorbitol, malvitol, trehalose, raffinose, xylitol, mannitol, polyethylene glycol, propylene glycol, polyglycerin, cholesterol, squaline, fatty acids, octyldodecanol, myristyl alcohol, myristyl lactate, urea, lanolin, lactic acid, esters such as isopropyl stearate, isopropyl myristate, isopropyl palmitate, light liquid paraffin, cetearyl alcohol, lanolin derivatives, mineral oil, petrolatum, cetyl esters, wax, cholesterol, glycerol monostearate, lecithin, isopropyl laurate and the like and combinations thereof.

Permeation enhancers may be incorporated in the pharmaceutical composition of the present invention for rectal administration for delivery of the active ingredient to the mucosal surface. Most types of enhancers are detergents that include: sodium glycocholate, sodium taurocholate, polysorbate 80, sodium lauryl sulfate, lauric acid, and various alkyl glycosides or combinations thereof. Other examples of enhancers include: dextrins (cyclodextrin, dextran sulfate), fatty acids (phosphatidylcholine, lysophosphatidylcholine), heterocyclic compounds (azone), and small molecules (benzalkonium chloride, cetyltrimethylammonium bromide) and combinations thereof.

Suitable mucoadhesives may be used in the pharmaceutical composition of the present invention for rectal administration to improve local retention of mucosally delivered active ingredient.

Mucoadhesive compounds are primarily synthetic or natural polymers that can adhere to the wet mucosal surface. These include synthetic polymers such as, but not limited to monomeric alpha cyanoacrylate, polyacrylic acid, hydroxypropyl methylcellulose, and poly methacrylate derivatives or combinations thereof. Glue-like polymers include epoxy resins and polyurethanes. Naturally occurring mucoadhesives include chitosan, hyaluronic acid and xanthan gum and combinations thereof.

Suitable emulsifiers include, but are not limited to, straight chain or branched fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block copolymers, and combinations thereof.

Suitable suspending agents include, but are not limited to, alginic acid, bentonite, carbomer, carboxymethylcellulose and salts thereof, colloidal oatmeal, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof. The antioxidant is preferably present in an amount of from about 0.01% to about 10% w/w based on the total weight of the pharmaceutical composition.

Suitable chelating agents include, but are not limited to, EDTA, disodium edetate, trans-1,2-diaminocyclohexane-N,N,N′,N′-tetraaceticacid monohydrate, N,N-bis(2-hydroxy ethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosphonic acid), 0,0′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphonic acid), 7,19,30-trioxa-1,4,10,13,16,22,27,33-octaazabicyclo[111,11,1]pentatriacon-tane hexahydrobromide, triethylenetetramine-N,N,N′,N″,N′″,N′″-hexaacetic acid, or combinations thereof. The chelating agents is preferably present in an amount of from about 0.01% to about 5% w/w based on the total weight of the pharmaceutical composition.

Preservatives can be used to prevent the growth of fungi and other microorganisms. Suitable preservatives include, but are not limited to, benzoic acid, sorbic acid, butylparaben, ethyl paraben, methyl paraben, propyl paraben, sodium benzoate, sodium propionate, benzalkonium chloride, benzethonium chloride, benzyl alcohol, cetylpyridinium chloride, chlorobutanol, phenol, phenylethyl alcohol, thimerosal, or combinations thereof. The preservative is preferably present in an amount from about 0.01% to about 2.0% w/w based on the total weight of the pharmaceutical rectal foam composition.

The present invention provides a method of treating or maintenance of remission of Clostridium difficile associated infections by administering pharmaceutical compositions for rectal administration comprising fidaxomicin to patients in need thereof.

The present invention further provides a pharmaceutical composition for rectal administration in the form of a foam comprising fidaxomicin for the use in the treatment of Clostridium difficile associated infections.

Preferably, the present invention further provides a pharmaceutical composition for rectal administration in the form of a foam comprising fidaxomicin for the use in the treatment of Clostridium difficile associated infections, wherein the infection comprises diarrhea.

Preferably, the present invention further provides a method of treating or maintenance of remission of Clostridium difficile associated infections by administering pharmaceutical compositions for rectal administration in the form of a foam comprising fidaxomicin to patients in need thereof, wherein the infection comprises diarrhea.

The following examples are for the purpose of illustration of the invention only and are not intended in any way to limit the scope of the present invention.

### EXAMPLES

### Example 1

| Sr. No. | Ingredients | Qty/Unit (% w/w) |
|---|---|---|
| 1. | Fidaxomicin | 0.2% |
| 2. | Disodium Edetate | 0.30% |
| 3. | Sodium Metabisulphite | 1.00% |
| 4. | Emulsifying Wax | 1.00% |
| 5. | Propellant | 3.75% |
| 6. | Propylene Glycol | q.s. to 100% |

Process:

(1) Propylene glycol was heated and emulsifying wax was dissolved.
(2) Sodium metabisulphite and disodium edetate were dispersed in the solution obtained in step (1).
(3) Fidaxomicin was dispersed in the solution obtained in step (2) to form a uniform suspension.
(4) The suspension obtained in step (3) was filled in a container and charged with the propellant.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants; reference to a “cosolvent” refers to a single cosolvent or to combinations of two or more cosolvents, and the like.

1. A pharmaceutical composition for rectal administration in the form of a foam comprising fidaxomicin.

2. A foamable pharmaceutical composition for rectal administration comprising fidaxomicin.

3. The pharmaceutical composition according to claim 1, wherein fidaxomicin is in the form of a pharmaceutically acceptable derivative thereof.

4. The pharmaceutical composition according to claim 3, wherein the pharmaceutically acceptable derivative of fidaxomicin is a salt, solvate, complex, hydrate, isomer, ester, tautomer, anhydrate, enantiomer, polymorph or prodrug.

5. The pharmaceutical composition according to claim 1, wherein fidaxomicin is present in an amount of from about 0.01% w/w to about 10% w/w based on the total weight of the composition, optionally from about 0.5% w/w to about 8% w/w based on the total weight of the composition.

6. The pharmaceutical composition according to claim 1, further comprising one or more pharmaceutically acceptable excipients which are selected from the group comprising: propellants, vehicle, emollient and/or humectants, pH adjusting agent, surfactants, emulsifiers, foaming agents, fatty alcohol, preservatives, chelating agents, antioxidants, suspending agents, thickening agents, lubricants, permeation enhancers, suspension-forming agents, mucoadhesive agents, mineral oils or combinations hereof.

7. The pharmaceutical composition according to claim 6, wherein the vehicle comprises an aqueous, non-aqueous or a hydro-alcoholic vehicle.

8. The pharmaceutical composition according to claim 7, wherein the aqueous vehicle comprises water soluble alkanols, ethanol, polyalcohols, propylene glycol, glycerol, polyethyleneglycol, polypropylene glycol, propylene glycol glyceryl esters or combinations thereof.

9. The pharmaceutical composition according to claim 7, wherein the non-aqueous vehicle comprises vegetable oils, olive oil, injectable organic esters, ethyl oleate or combinations thereof.

10. The pharmaceutical composition according to claim 6, wherein the vehicle is present in an amount of from about 10% to 95% w/w based on the total weight of the composition; preferably from about 20% to 85% w/w based on the total weight of the composition.

11. The pharmaceutical composition according to claim 6, wherein the emulsifier is present in an amount of from about 0.5% to about 10% w/w based on the total weight of the composition.

12. The pharmaceutical composition according to claim 6, wherein the propellant comprises isobutane, n-butane, propane, CFC, hydrocarbons; chlorofluorocarbons (CFCs); hydrochlorofluorocarbons (HCFCs); hydrofluoroalkanes (HFAs), HFA 134a and HFA 227 or combinations thereof.

13. The pharmaceutical composition according to claim 6, wherein the propellant is present in an amount of from about 0.05% to 20% w/w based on the total weight of the composition, optionally from about 1% to 10% based on the total weight of the composition.

14. The pharmaceutical composition according to claim 6, wherein the surfactant comprises anionic surfactants, non-ionic surfactants, cationic surfactants, amphoteric surfactants or combinations thereof.

15. The pharmaceutical composition according to claim 6, wherein the surfactant is present in an amount of from about 0.1% to about 10.0% w/w based on the total weight of the composition; optionally from about 0.1% to about 8.0% w/w based on the total weight of the composition.

16. The pharmaceutical composition according to claim 1, further comprising at least one or more additional active ingredient selected from one or more of anti-inflammatory agents, steroids, antibiotics, anti-fungal agents, analgesics, anti-neoplastic agents, antivirals and anaesthetics.

17. The pharmaceutical composition according to claim 1, wherein the pH of the rectal foam is in the range of from about 4 to 8.

18. The pharmaceutical composition according to claim 1, wherein the foam is in the form of a stable non-aqueous foam, a stable aqueous foam, an evanescent or a quick breaking non-aqueous foam or an evanescent or a quick breaking aqueous foam.

19. A process of manufacturing a pharmaceutical composition for rectal administration in the form of a foam comprising fidaxomicin, the process comprises: heating a vehicle and adding an emulsifier to the vehicle, optionally along with other pharmaceutically acceptable excipients followed by adding fidaxomicin to obtain a blend; and optionally further comprising filling the blend into a container and charging it with a propellant.

20. The process according to claim 19, wherein the vehicle comprises an aqueous, non-aqueous or a hydro-alcoholic vehicle.

21. The process according to claim 19, wherein the emulsifier is present in an amount of from about 0.5% ti about 10% w/w based on the total weight of the composition.

22. The process according to claim 19, wherein the propellant comprises isobutane, n-butane, propane, CFC, hydrocarbons; chlorofluorocarbons (CFCs) hydrochlorofluorocarbons (HCFCs); hydrofluoroalkanes (HFAs), HFA 134a and HFA 227 or combinations thereof at optionally the propellant is present in an amount of from about 0.05% to 20% w/w based on the total weight of the composition.

23. An aerosol canister for a pharmaceutical composition as defined in claim 1, which is capable of forming a foam, comprising a housing containing under pressure the pharmaceutical composition; means for measuring a metered dose of the composition from the canister for administration to a patient in need thereof; and optionally comprising an applicator device for rectal administration.

24. The aerosol canister according to claim 23, wherein the means for measuring a metered dose of the pharmaceutical composition from the canister is a valve or a pump dome.

25-26. (canceled)

27. A method of treating or maintenance of remission of infections caused by Clostridium difficile comprising administering an effective amount of a pharmaceutical composition according to claim 1 to a subject in need thereof.

28. The method according to claim 27, wherein the infection comprises diarrhea.

29. (canceled)
